(19) United States
(12) Patent Application Publication
(10) Pub. No.: US 2026/0354706 A1
(43) Pub. Date: Nov. 12, 2026

(54) Fine Wire Muscle Interfaces

(71) Applicant: Massachusetts Institute of Technology, Cambridge, MA (US)

(72) Inventors: Cameron Roy Taylor, Edina, MN (US); Hugh M. Herr, Concord, NH (US)

(21) Appl. No.: 19/139,268

(22) PCT Filed: Dec. 14, 2023
(86) PCT No.: PCT/US2023/084147

§ 371 Date: Jun. 13, 2025

Related U.S. Application Data
(60) Provisional application No. 63/387,528, filed on Dec. 15, 2022.

(51) Int. Cl. A61B 5/11; A61B 5/00; A61B 5/053; A61B 5/395

(52) U.S. Cl. CPC A61B 5/1107; A61B 5/053; A61B 5/395; A61B 5/6828

(57) ABSTRACT

Devices, systems, and methods described herein relate to a muscle interface and methods of use. The muscle interface may include an implant comprising a plurality of fine wires configured to be disposed in muscle tissue and to detect an electrical impedance at the muscle tissue; and a processor configured to determine a state of the muscle tissue based on the detected electrical impedance.

### RELATED APPLICATION

This application claims the benefit of U.S. Provisional Application No. 63/387,528, filed on Dec. 15, 2022.

The entire teachings of the above application are incorporated herein by reference.

### BACKGROUND

Muscle tissue drives nearly all movement in the animal kingdom, providing power, mobility, and dexterity. Technologies for measuring muscle tissue motion have significantly advanced our understanding of biomechanics and can enable new control modalities for prosthetic and exoskeletal control. One candidate for measuring muscle motion is image-based ultrasound, which allows non-invasive muscle imaging, but currently has poor muscle fascicle visibility, and thus is difficult to use for muscle fascicle length tracking.

Technologies such as sonomicrometry, fluoromicrometry, and magnetomicrometry provide improved accuracy in muscle fascicle length tracking but require an implantation procedure performed by a clinician to place the devices within the muscle. As society progresses forward, it is important for us to be able to have medical sensing solutions which are both minimally-invasive and can be implemented at home.

### SUMMARY

Aspects of inventive concepts relate to a muscle interface. The muscle interface may include an implant comprising a plurality of fine wires configured to be disposed in muscle tissue and to detect an electrical impedance at the muscle tissue; and a processor configured to determine a state of the muscle tissue based on the detected electrical impedance.

In various embodiments, the implant is configured to detect at least two electrical impedances at the muscle tissue.

In various embodiments, the implant is configured to detect an electrical impedance between each combination of two fine wires of the plurality of fine wires.

In various embodiments, the determined state comprises at least one of length, velocity, or deformation of the muscle tissue.

In various embodiments, the implant is further configured to detect an electromyographic signal at at least one wire of the plurality, at an additional wire of the implant, or at a combination thereof.

In various embodiments, the processor is further configured to determine an activation level of the muscle tissue based on the detected electromyographic signal.

In various embodiments, the muscle interface further comprises a controller electrically coupled to the processor.

In various embodiments, the implant is further configured to electrically stimulate the muscle tissue via at least one wire of the plurality, at an additional wire of the implant, or at a combination thereof.

In various embodiments, the controller is further configured to generate a muscle stimulation signal for the implant based on the determined state.

In various embodiments, the controller is further configured to generate a control signal for an actuator of a wearable robotic device based on the determined state.

Aspects of inventive concepts relate to a method of interfacing with a muscle. The method may include detecting an electrical impedance at a muscle tissue with an implant comprising a plurality of fine wires disposed in the muscle tissue; and determining a state of the muscle tissue based on the detected electrical impedance.

In various embodiments, the method further includes detecting at least two electrical impedances at the muscle tissue.

In various embodiments, the method further includes detecting an electrical impedance between each combination of two fine wires of the plurality of fine wires.

In various embodiments, the determined state includes at least one of length, velocity, or deformation of the muscle tissue.

In various embodiments, the method further includes detecting an electromyographic signal at at least one wire of the plurality, at an additional wire of the implant, or at a combination thereof.

In various embodiments, the method further includes determining an activation level of the muscle tissue based on the detected electromyographic signal.

In various embodiments, the method further includes electrically stimulating the muscle tissue via at least one wire of the plurality, at an additional wire of the implant, or at a combination thereof.

In various embodiments, the method further includes generating a muscle stimulation signal for the implant based on the determined state.

In various embodiments, the method further includes generating a control signal for an actuator of a wearable robotic device based on the determined state.

Aspects of inventive concepts relate to a method for controlling drop foot. The method may include monitoring a length of a tibialis anterior using a fine wire impedance myography device, the device including fine wires; determining if the length of the tibialis anterior exceeds a reference length; stimulating the tibialis anterior during an early swing phase of gait using the fine wires of the fine wire impedance myography device; and ending the stimulation when the swing phase is more than a given percentage complete.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows a side view of an example embodiment of a Percutaneous Fine-Wire Impedance Myography device, in accordance with aspects of inventive concepts.

FIG. 2 shows an example embodiment of a device for Percutaneous Fine-Wire Impedance Myography (fwIM) and Functional Electrical Stimulation Combined for Correction of Drop foot, in accordance with aspects of inventive concepts.

FIG. 3 shows an example embodiment of a device for Transcutaneous Fine-Wire Impedance Myography, in accordance with aspects of inventive concepts.

FIG. 4 shows a State Transition Diagram for Drop foot Control via Combined Fine Wire Impedance Myography and Functional Electrical Stimulation, in accordance with aspects of inventive concepts.

### DETAILED DESCRIPTION

A description of example embodiments follows.

The teachings of all patents, published applications and references cited herein are incorporated by reference in their entirety.

Techniques for using fine wires to track tissue lengths and velocities are described herein. In various embodiments, these wires can be less than 1 mm in diameter, though ideally less than 0.1 mm in diameter. These techniques can be used for tracking the length and velocity of muscle tissue. The implantation of fine wires can be performed simply, for example, by at-home-implantable continuous glucose monitors (CGMs), making the strategy minimally-invasive, or due to the minimally-invasive nature of fine wires, many fine wires can be implanted at once, whether percutaneously or as part of an implanted neural technology (e.g., eOPRA, Ripple or the like). Percutaneous fine wires can be used without discomfort to the patient and can be easily made biocompatible. Systems, device, and/or methods described herein can be made portable with wired or wireless transmission and in combination with a computer for prosthetic and exoskeletal control. In some example embodiments, a motor controller can directly control a robotic joint using muscle motion tracked in each muscle of an agonist-antagonist pair. More generally, inventive concepts described herein have broad implications for human-machine interfacing. In describing example embodiments herein, the features and usage described in connection with a computer also apply to a processor. In describing example embodiments herein, the features and usage described in connection with a processor also apply to a computer.

The ability for a technique to track additional muscles and to work in combination with muscle activation signals enables a range of new strategies for human-machine interfacing. Muscle length and velocity signals are different from, and complementary to, muscle activation signals, such as the signals from electromyography (EMG). While EMG provides a measure of muscle activation, which results more directly from neural commands, muscle length and velocity give information on the shape of the muscle, which in turn can refine our understanding of muscle physiology during a given motion task. Indeed, the combination of muscle motion and activation will allow for increased physiological understanding in new contexts and improved human machine interfaces.

### Applications

Drop foot (also called foot drop) is a sometimes temporary, but more often permanent disability where a patient is unable to lift the front of their foot (i.e., dorsiflex their ankle). Drop foot can be caused by nerve injury, such as a traumatic-injury compression of the peroneal nerve, or injury to the peroneal nerve during a surgery. Furthermore, drop foot can result from a nerve root injury in the spinal cord, or a muscle, brain, or spinal cord disorder causing muscle weakness or neural signaling pathology. Drop foot most typically results from a number of disease conditions such as polio, Charcot-Marie-Tooth disease, amyotrophic lateral sclerosis (ALS), multiple sclerosis, and stroke. The dominant solution addressing drop foot is the use of an ankle-foot orthosis (AFO) that holds the ankle in a neutral position so that 1) the forefoot does not slap on the ground during the early stance phase of gait, and 2) the toes of the affected leg can clear the ground during the swing phase of gait. This passive solution does not restore biomimetic walking speed and terrain functionality of the ankle-foot complex, as it is limited to a fixed ankle stiffness designed to mitigate foot slap after heel strike and enhance foot clearance throughout the swing phase.

To address drop foot more comprehensively, a myoneural interface that measures muscle motion can use an artificial muscle stimulation controller to determine when to stimulate, using the muscle motion sensing as feedback signals for closed-loop control. Such a technological platform can, for example, use gastrocnemius muscle sensing to get toe-off information, combined with sensing and stimulation of the tibialis anterior, and further, optionally, can be combined with the use of an inertial measurement unit, insole pressure measurement device, or other sensing modalities.

This strategy for alleviating drop foot can be similarly applied to other neuropathological cases involving different affected muscles to provide increased fidelity in the muscle control via artificial stimulation, whether this artificial stimulation is provided via electrical, optical, electromagnetic, or some other means. Neuropathologies include but are not limited to spinal cord injury, stroke, polio, Charcot-Marie-Tooth disease, amyotrophic lateral sclerosis (ALS), and/or multiple sclerosis.

The tracking of muscle motion can also be used for the control of an active or quasi-passive prosthesis, especially in the case of a person who has an osseointegrated connection to their prosthesis. Further, muscle motion can be used to control a powered orthosis or exoskeleton for any of the above diseases or others.

Fine-wire muscle tracking can be performed via the insertion of one or more wires into tissue and monitoring a signal that describes the movement of the one or more wires. The techniques described herein will be described from the perspective of tracking the motion of biological tissue, such as animal and human tissue, such as muscle tissue, but can be applied more generally to other tracking applications outside of biology, such as in tracking motion or stress of other natural or artificial materials, such as rock or concrete.

Fine-wire muscle tracking works by tracking either the motion or state of each wire, or by tracking the relative motions or states between wires. For instance, in some example embodiments, the distance between the ends of the inserted fine-wires affects the signal that is used for the tracking of the muscle motion. In some example embodiments, the distance between the wires themselves (not only the tips, but the body of the wires) affects the signal used for tracking. In some example embodiments, it is the bend of the wires that provides the muscle motion signal. In some example embodiments, it is the presence or difference in presence of particular chemicals, such as calcium ions, around the one or more wires that is used to determine the muscle motion or muscle activation.

In all of these cases, the use of a single wire or a pair of wires can be expanded into the use of a network of implanted wires. The above example embodiments can be combined in any combination.

### Insertion Strategy

While percutaneous fine-wires may be inserted by a clinician, these fine-wire tissue tracking strategies are designed with the potential to be inserted percutaneously with a method that can allow the patient to insert the fine wires at home, similar to how a CGM device, such as how the Dexcom G6 allows at home insertion of fine wires. A device may then also be attached to the surface of the skin to cover the insertion point and may also have any needed electronics for power and communication embedded within the device. The device may be implanted via palpation, via ultrasound (especially via portable ultrasound), via virtual or augmented reality guidance, via trial and error (such as repeated insertion or via insertion of more wires than necessary and only keeping the relevant wires inserted or only using the relevant wires as signals), or via a combination of any of these or some other guidance, and this guidance may also be informed by other imaging modalities such as magnetic resonance imaging or computed tomography.

For percutaneous wire insertion, the insertion may be performed using a needle which is inserted having the fine wire(s) through the bore of the needle, after which the needle is removed, and the fine wire(s) are left inserted. A bend in the fine wire(s) can help with holding the fine wire(s) in place. Alternatively, a rigid or semi-rigid needle can be used to thread the fine wire(s) by piercing the insertion site next to the fine wire(s), after which the needle can be removed, and the fine wire(s) stay in place. As another alternative, a jet of fluid such as water can be used to create the insertion path and push the fine wire(s) into the path at the insertion site. As another alternative, the fine wire(s) can be made of a material which is rigid at the time of insertion and becomes flexible after insertion, using chemical, thermal, optical, electromagnetic, or other means.

While in some example embodiments fine-wires are inserted percutaneously (through skin), the wires can alternatively be wired through a skin port, through bone or prosthesis (such as in the case the use of an e-opra device), or via an implanted device.

Further, the implanted wires can be powered transcutaneously via wireless power to remove the need for a percutaneous wire. It will be understood by those of ordinary skill in the art that the fine-wire inventive concepts described herein can be used as a component of an implanted neural system. In some example embodiments, for example, an osseointegrated implant with fine wires passing through the implant can employ the muscle motion sensing inventive concepts described herein. Here one or more fine wires can be placed into each muscle of an amputated residuum to measure muscle lengths, velocities, and/or activations for high fidelity external prosthesis joint control. These implanted muscle wires can then pass into a hole within the residual bone and then through the osseointegrated bone implant and finally into the external prosthetic controller.

### Materials and Coatings

Standard biomedical device materials and coatings can be used that allow the electrical, mechanical, and/or optical properties for the fine-wire devices described herein, such as gold, platinum, parylene and polydimethylsiloxane. The base materials need not be biocompatible, though ideally would be. If the base material is biocompatible and a coating is not needed for the functioning of the device, the device may not use a coating. Fine wires that are currently used for other interfacing strategies, such as fine-wire EMG, can be used for these techniques.

### Methods of Operation

### Fine-Wire Impedance Myography

In some example embodiments, impedance myography (IM) is used to observe spatial deformations of muscle via surface electrodes and is a type of electrical impedance tomography (EIT). The use of multiple frequencies (bioimpedance spectroscopy) reveals additional information about the observed tissues since cellular membranes can only be penetrated at higher frequencies. IM can be combined with surface electromyography (EMG) to improve muscle sensing, for instance for prosthetic control via residual muscles. IM typically works by either injecting a current and monitoring voltage or by controlling a voltage and monitoring current, and the relationship between the current and voltage provides information about the impedance of the material through which it travels.

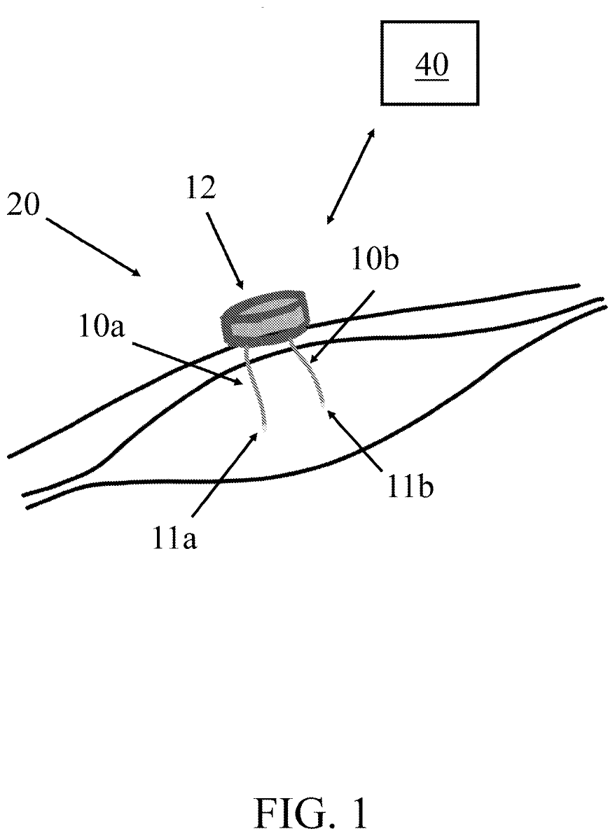

In some example embodiments, implanted electrodes, such as those used in fine-wire EMG, can employ the strategy of IM to measure muscle tissue lengths (see FIG. 1). We call this strategy fine-wire impedance myography (fwIM). Fine-wire impedance myography can use standard frequencies for impedance myography such as 10 kHz or 50 kHz, or can be much lower or higher, from 0 Hz to 5 MHz or more. One or more frequencies can be used simultaneously for the sensing. When using lower frequencies, the amount of intracellular membrane through which the current travels is what affects the signal, while at higher frequencies, the current also travels through the cells. Therefore, different information is able to be found at the different frequencies, and this information can be used to find signals such as tissue length and velocity, but may also be used to find signals such as temperature, pressure, hydration, activation, ion concentrations, stiffness, or force, or other measurable physical quantities of the muscle, which, including the length or velocity, we will refer to here very generally as the “state” of the muscle. These signals may be discovered in real-time or over time. The impedance measurements also need not be performed using sine waves, as any other waveform can also be used to determine the complex impedance of the muscle, such an impulse or impulse train, a square, trapezoid, triangle, or sawtooth wave, white noise, and so on. The waves can be balanced or imbalanced (in other words, their integrals may be zero or nonzero). Because the impedance measured is a complex value, the real and/or the imaginary part of the impedance signal can be used to determine the state of the muscle. For instance, the magnitude or phase of the impedance can be used as a signal. In addition, the real and/or imaginary part of the one or more complex impedance frequencies and signals that are derived from these can be used as a single-or multi-dimensional lookup table (for instance, using nearest neighbor search techniques) to determine the actual muscle state signal of interest. Alternatively, a statistical or machine learning tool can be used to create a map or function from the complex impedance values at the one or more frequencies used (likely each driven by a separate oscillator). Examples of algorithms that can be used to perform this mapping include, but are not limited to, linear or logistic regression, decision trees, support vector machines, naive Bayesian models, k-nearest neighbors, k-means, random forests, dimensionality reductions, or gradient boosting algorithms (such as GBM, XGBoost, LightGBM, or CatBoost). Alternatively, or in addition, a biophysical model of the muscle physiology, for instance, of the complex impedance of the intracellular matrix and of the cells, can be utilized in forming informed determination about the deformation of the muscle or other metrics of muscle state. In creating the mapping from the complex impedance to the muscle of interest, a calibration technique can be employed that involves the person moving their muscle in a way that best mimics how the muscle will be used with the fine-wire muscle interface, ideally while measuring the muscle activity to get a reference for the physical quantity of interest, then creating a mapping that can be used to predict the physical quantity of interest from the complex impedance. For instance, ultrasound may be used to get fascicle length or velocity, and electromyography might be used to get muscle activation. A muscle biophysical model can then be used to estimate muscle-tendon force.

Alternatively, inertial measurement units (IMUs) may be used to estimate limb kinematics, and a pressure/force insole to get external limb forces. Using these data, an inverse dynamics calculation might be used to estimate muscle length, speed, or force. Mapping to one or more of these muscle quantities (activation, length, velocity, force) from the complex impedance measurements during a calibration routine can then be performed.

FIG. 1 shows a side view of an example embodiment of a Percutaneous Fine-Wire Impedance Myography device 20, in accordance with aspects of inventive concepts. In this example embodiment, two insulated wires 10a, 10b (blue) with bare conductor tips 11a, 11b (yellow) are inserted percutaneously into muscle. An electronic patch or electronic chip 12 (gray) houses the electronics. In some example embodiments, such as the one shown in FIG. 1, the electronics comprises at least one or more of a computer, battery, current-driver, oscillator, voltage-sensor, or communications hardware. In some embodiments, such as the one shown in FIG. 1, the patch or chip 12 also serves to prevent infection at the site or movement of the implanted fine wires 10a, 10b. The chip 12 remains in place due to an adhesive, either between the chip and the skin and/or over the chip to affix it to the skin. The implant can be used short-term or long-term. In alternative embodiments, the device 20 may comprise a different number of insulated wires 10.

In the embodiment shown in FIG. 1, the computer/processor 40 is at an external location outside of the chip 12. In alternative embodiments, the electronic chip 12 houses the computer/processor 40. In the embodiment shown in FIG. 1, the computer/processor 40 communicates with the electronic chip 12 wirelessly. In alternative embodiments, the computer/processor communicates with the electronic chip 12 with a wired connection.

Fine-wire IM can be performed using the fine wire electrodes used for fine-wire EMG, which are insulated everywhere except at the tips. The wires used can be bipolar or monopolar (either two wires or one wire inserted at each site), and one of the two bipolar electrodes can be used (for instance, solely inserting one of the two wires or inserting both wires and removing one of them). Fine-wire IM can also be combined with fine-wire EMG, performing both muscle impedance and muscle activation measurements simultaneously.

Such a measurement strategy can allow for calculation of muscle force directly via the combined information from, for instance, muscle length, velocity, and activation. This strategy can, for instance, provide highly accurate, minimally invasive control of a prosthesis or control of a robotic exoskeleton to restore natural biomechanics of a neuromechanically-impaired biological limb.

The fwIM can be performed with a single pair of electrodes or with many electrodes at once. The most minimally-invasive case for two-wire fwIM is a pair of electrodes implanted similar to how the single wire of a continuous glucose monitor (CGM) wire is implanted, as in the implantation strategy for the Dexcom G6, but implanting two wires at different implantation sites. These wires can be implanted at different times or simultaneously, and they can be implanted with different hardware or with a single piece of hardware, and they can be permanently connected or electrically connected after implantation via wire or wireless connectivity.

Examples of systems that can be used or modified for use for fwIM are the MAX30001 Ultra-Low-Power, Single-Channel Integrated Biopotential (ECG, R-to-R, and Pace Detection) and Bioimpedance (BioZ) analog front-end (AFE) solution for wearable applications by Maxim Integrated (Analog Devices), the MAX30002 Ultra-Low Power, Small Package, Single Channel Integrated Bioimpedance (BioZ) AFE for Fitness and Medical Applications by Maxim Integrated (Analog Devices), the MFIA 500 kHz/ 5 MHZ Digital Impedance Analyzer and Precision LCR Meter by Zurich Instruments, the MFLI 500 kHz/5 MHz Lock-in Amplifier by Zurich Instruments, the ADUCM350 Configurable Impedance Network Analyzer & Potentiostat with Integrated Cortex M3 Core by Analog Devices, the AD59331 MSPS, 12-Bit Impedance Converter, Network Analyzer by Analog Devices, and the AD5934 250 kSPS, 12-Bit Impedance Converter, Network Analyzer by Analog Devices.

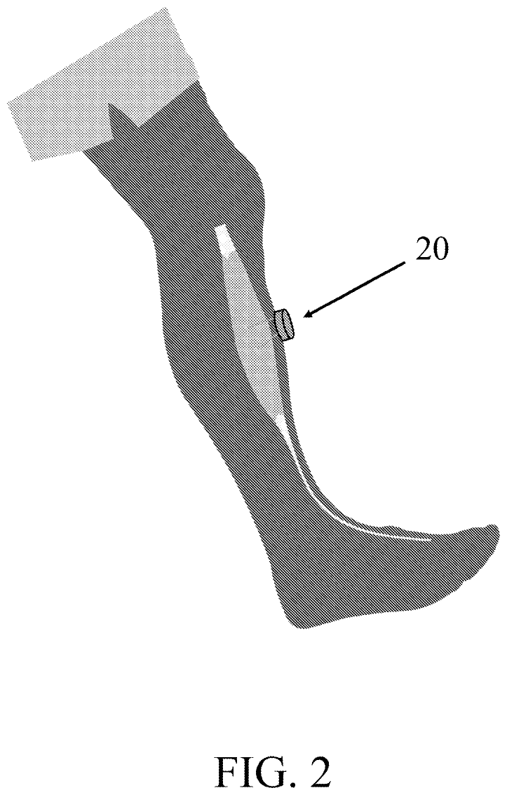

Combined fwIM with functional muscle stimulation can be used to design an electrical or optical muscle stimulation paradigm that controls muscle length, velocity, or force in a closed-loop manner under computer control. In such a framework, at least one of a muscle length, speed, or force can be estimated in real time using fwIM, and used as closed-loop feedback signals for a muscle stimulation controller. In some example embodiments, optical or electrical muscle stimulations can be applied in an updating manner based upon the at least one measurement of muscle length, velocity, or force in real time. Further, in the case of weak musculature, such a combination of fwIM and functional muscle stimulation can minimize muscular fatigue or amplify muscle force and power output (see FIG. 2).

Alternatively, fwIM can be used to monitor spatial deformation of the muscle while the source nerve is stimulated. For all these embodiments, closed-loop control can be enabled by stimulating muscle or nerve either through the fwIM wires or through some other artificial stimulation technique while monitoring the fwIM signals. For instance, muscle length setpoint control can be performed via this strategy. Alternatively, muscle force setpoint control can be performed via this strategy. Here fwIM can measure muscle length, velocity or EMG. These signals can then be used to estimate muscle force using a biophysical muscle model (e.g., Hill muscle model). This measured biological muscle force can then be used to estimate biological joint torque using a joint moment arm estimation. This biological torque can then serve as a setpoint for an exoskeletal controller where exoskeleton torque is functionally related to the biological torque. Any one of these closed-loop control strategies can be combined with a mix of sensing and/or stimulation on multiple muscles simultaneously, where not all muscles are necessarily sensed or stimulated, and this strategy can be combined with other sensing strategies, including but not limited to ultrasound, pressure insoles, IMUs, or nerve cuffs.

FIG. 2 shows an example embodiment of a device 20 for Percutaneous Fine-Wire Impedance Myography (fwIM) and Functional Electrical Stimulation Combined for Correction of Drop foot, in accordance with aspects of inventive concepts. In this particular example, the fwIM device 20 (as seen in FIG. 1) is shown affixed to the front of the leg with the fine wires 10a, 10b located in the tibialis anterior muscle.

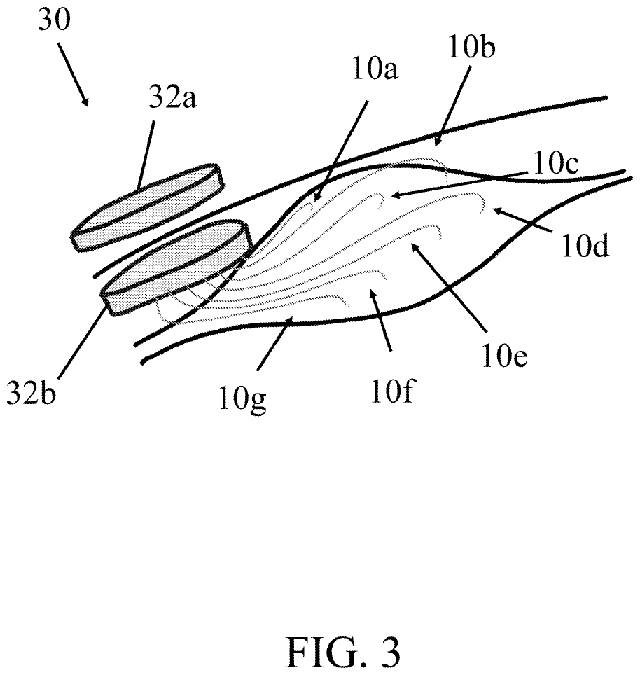

The fwIM technology can also be performed with any number of additional wires to more easily sense multi-modal three-dimensional information about a muscle, looking at the relationship between the wires pairwise or combining voltage and/or current input and voltage and/or current sensing in new combinations between the wires (see FIG. 3). For instance, the wires may be inserted in such a way that some wires get farther apart in the muscle while some get closer together during a particular type of muscle contraction. As a particular case of this instance, if four wires are inserted and two wires get closer to one another while two get farther apart, in such a case a full-bridge Wheatstone bridge is created, and this can enable a strategy of stimulation and sensing where a constant input voltage is introduced at two leads, which is modulated by the resistances to provide, for instance, a zero output voltage at the two other leads when the muscle is not contracted, a positive (or negative) voltage when the muscle is contracted, and a negative (or positive) voltage when the muscle is stretched. Other configurations can of course be provided, such as, for example, a quarter bridge configuration where two inserted fine wires constitute the active part of the Wheatstone bridge while the remaining leads are passive. Of course, many other circuit configurations, such as other bridge configurations can be used, employing multiple passive or quasi-passive circuit elements within or outside the body.

FIG. 3 shows an example embodiment of a device 30 for Transcutaneous Fine-Wire Impedance Myography, in accordance with aspects of inventive concepts. In this example, electronic chips or patches 32a, 32b (each at least similar to the chip or patch 12 described in connection with FIG. 1) are located both outside and inside the body, each having some subset of the electronics that are described in connection with the percutaneous embodiments of inventive concepts, but the power and communications are delivered transcutaneously via magnetic fields, sound or vibration, ultrasound, light, electromagnetic waves, or via some other means. The one or more wires are connected to the chip that is within the body. This setup or one similar to it can be used for fwIM or for any of the other embodiments of inventive concepts described herein. For the particular case of fwIM, in some embodiments, the current driver, oscillator, and voltage sensing can be in the implanted chip, along with a battery, and a computer and battery can be in the externally-mounted chip. In this case, the externally mounted chip can be affixed to the skin or can be affixed to clothing, since perfect and consistent alignment is no longer necessary.

In the embodiment shown in FIG. 3, the computer/processor 40 can be at an external location outside of the chips 32a, 32b. Alternatively, at least one of the electronic chips 32a, 32b can house the computer/processor 40. In various embodiments, the computer/processor 40 communicates with at least one of the electronic chips 32a, 32b wirelessly. In alternative embodiments, the computer/processor communicates with at least one of the electronic chips 32a, 32b with a wired connection.

In the example embodiment shown in FIG. 3, the device 30 comprises seven wires 10a-10g. In alternative embodiments, the device 30 of FIG. 3 or the device 20 of FIG. 1 may comprise a different number of wires 10. In various embodiments, the impedance between each combination of two wires may be measured. For example, device may measure the impedance between the first wire 10a and the second wire 10b, the first wire 10a and the third wire 10c, etc. Similarly, the device may measure the impedance between the second wire 10b and the third wire 10c, the second wire 10b and the fourth wire 10d, etc. In various embodiments, impedance measurements, and/or other types of measurement described herein, may be performed for each combination of two wires. In various embodiments, impedance measurements, and/or other types of measurement described herein, may be performed for a subset of each combination of two wires.

It is also possible to perform fwIM with a single lead. This allows for sensing the impedance at the site of the implantation, though a distance signal is less straightforward from a single lead. In such a case, a ground lead can be placed on the surface of the skin to complete the circuit. Such a circuit may still allow for any of the above metrics to be measured, such as hydration, ion concentration, and so on.

The fwIM technique can be performed percutaneously as with fine-wire EMG using fine wires that pass through the skin boundary or via an osseointegrated implant, or the wires can be fully implanted with some electronics and powered and/or communicated across the skin boundary using a wireless signal transmission.

The wires may be introduced uninsulated, fully-insulated, or insulated everywhere except for at the tip (such as how the wires for fine-wire EMG are typically constructed). In some example embodiments, only the tip is left exposed, to provide a more direct relationship between the signal and the distance between the tips of the plurality of fine wires implanted in a muscle.

Though some of the electronics for impedance sensing, EMG sensing, and electrical muscle stimulation are similar, different electronic components are likely needed for each of these modes of interfacing with the muscle. Thus, in some example embodiments, the fine wires can be implanted leaving a minimal interface to the electrodes (for instance, solely access to the leads), and modular electronic components may be added. For instance, a patch above a pair of inserted fine wires can accommodate a stack where first an impedance sensing electronic chip is placed over the patch, which provides access to the same electrode leads above it, and above it is stacked an EMG sensing electronic chip, which provides access to the same electrode leads above it, and an electrical muscle stimulation chip is stacked above that. Thus, each modular component can either access the leads directly through a minimal interface or through one of the other modular components. The minimal component that lies over the inserted fine wires (for instance, the patch over the fine wires, which can also serve to protect against infection), can itself contain electronics that serve one or more of these functions. A modular component can serve to sense impedance at one frequency or more, and multiple modular components sensing impedance at different frequencies can be employed simultaneously. Because impedance sensing, EMG sensing, and electrical muscle stimulation can occur at different frequencies and perform electrically compatible functions, all of these functions can be performed simultaneously, though temporal switching can be performed so that some functions occur at different times (for instance, sensing EMG at a different time than electrically stimulating the muscle, to minimize the issues which can accompany sensing artificially induced voltages in the muscle).

### Other Strategies for Fine-Wire Interfacing With Muscle

The basic principle of fine-wire muscle interfacing is to use the positions of the tips of the fine-wires, which can be inserted percutaneously, or fully implanted with a wireless transmission, as a signal to determine muscle length, velocity, activation, and/or deformation. To this end, many other strategies can rely on this principle.

### Optical: Fiber Bragg Gratings

For instance, fiber Bragg gratings can be used to determine the stretch or bending of fine optical fibers. These cables can have multiple fiber Bragg gratings tuned to different optical frequencies along their length to get multiple signals out, and portable optical spectroscopy can be performed to interrogate the signal, after which a computer can interpret the signals out to monitor the state of the muscle. If needed, the optical fibers can be attached to one another to further facilitate bending measurements.

### Optical: Light Attenuation

Optical fibers can also be used to deliver light to the tip of a wire. Alternatively, light can be delivered to the tip of a wire or along the wire using electrical wiring and one or more small light-emitting diodes. When paired with a sensor either also implanted within the tissue or outside the body, the attenuation of the light can be used as a signal to determine distance. In addition, the phase and/or polarization of the light can be used as a signal to determine the state of the muscle. In particular, the optical sensor can be a small optical sensor at the end of a fine wire. Similar to the description above, one of more receivers can receive signals from each of one or more transmitters. Multiple frequencies of light from the infrared to ultraviolet wavelengths in the optical spectrum can be employed with embodiments described herein.

### Radio Frequency Attenuation

With a strategy similar to (and in some cases, compatible with) fwIM, radio frequency attenuation of an inserted wire can be used to monitor the state of the muscle. For instance, the resonance of an inserted wire can be used to determine information about the muscle state (particularly about hydration, though this would provide some information about muscle deformation and other physical quantities as well). In addition, the radio frequency attenuation between two inserted wires can be used. As a main instantiation of this strategy, the inserted fine wires can be viewed as monopole antennas, one can act as a transmitter, and the other can act as a receiver. The signals to be sent can be in the super low to super high frequency range (30 Hz to 30 GHz), and depending on the level of attenuation, the wires can be placed closer to one another when needed to ensure sufficient signal gets through. In the case of more than two wires, a single wire can act as the transmitter while the remaining wires act as receivers. In addition to monitoring the frequency attenuation, the receiving wires can also monitor the phase shift from the input signal. Fine wires can also be inserted in a loop, with either a single loop doubling back on itself, or two wires inserted as described in the primary fwIM technique described above can form a loop with the muscle tissue forming part of the loop.

### Acoustic

A well-known strategy for tracking muscle tissue deformation is called sonomicrometry. In sonomicrometry, piezoelectric crystals (typically ceramic) are implanted in muscle and are caused to vibrate using an electric current via percutaneous wires, and the time delay (and sometimes the phase) of the acoustic signal from one crystal to the next is used to determine the distance between the crystals, using the speed of sound traveling through the muscle tissue. In some example embodiments, tiny piezoelectric crystals may be used to enable the minimally-invasive percutaneous implantation of the wires and crystals, and the crystals may be set up to either vibrate tangent to the wire that connects to it or may vibrate orthogonal to the wire in one or all directions, to simplify the need for precise placement of the crystals.

Alternatively, instead of using the piezoelectric effect, the wire itself can be used as the medium for sound transduction. A material that is inherently resonant, such as metal, can be used, or this can be performed using a wire tube with a resonant cavity (an acoustic waveguide) filled with materials such as a gas, a liquid, a metal, or some other material which can carry an acoustic signal. The signal can be delivered along the wire via, for instance, a transverse or shear wave. The tip of the wires may be coated in something resonant as well, such as ceramic. The transmitting wires may be implanted while the receiving sensors are external (for instance, performing ultrasound measurements while the sounds are emitted in the ultrasound spectrum), or the receiving wires may be implanted while the transmitting acoustic emitters are external.

### Tiny Sensors (Active or Passive)

Advancing electronics due to commercial demand for sensors such as IMUs and computer hardware has made the development of small sensors a reality which continues to progress forward at a rapid pace. Due to the miniaturization of sensors, it is now possible, and where it is not yet performed, will soon be possible to place small sensors (less than 1 mm, though ideally less than 0.1 mm) at the end of or along fine wires. For instance, tiny magnetometers, accelerometers, and gyros can be used individually or with any subset to sense movement of a tissue site or differences in movement between multiple tissue sites.

These techniques can use single or multiple axes from a subset of these different sensors.

Further, a tiny magnetometer(s) inserted at the end of a fine wire can be paired with a magnetic field source, such as an implanted magnet or an externally-applied magnetic field, to track the position of the wire(s). Other physical quantities can be sensed similarly, such as the use of a temperature sensor, pressure sensor (such as though used in altimeters), or a bend sensor.

While, generally, embodiments of inventive concepts herein may use fine wires for interfacing with and sensing the muscle, the one or more tiny sensors can be powered and/or communicated with wirelessly, for instance, via electromagnetic or ultrasound waves.

### Chemical Sensing

Chemical sensing at the tip of or along the wire can be used to sense ion concentrations in the muscle to determine the action of a muscle as relayed via neural ion signaling. For instance, oxidation or reduction reactions with calcium that convert the presence of calcium into a voltage can be used to determine activation of the muscle. This is similar to the mechanism of operation of a CGM, but can be used for muscle state sensing and can be applied to human-machine interfacing as described earlier.

Such a strategy can also provide information about muscle hydration, an important metric, as it modulates the output power of muscle tissue.

Alternatively, one or more fine wires implanted may be fully or partially coated in materials that make them each an anode, a cathode, or partially anode and partially cathode. This anode cathode pair may be used to perform pH sensing of the muscle to interrogate the muscle state.

### Closed Loop Sensing and Stimulation

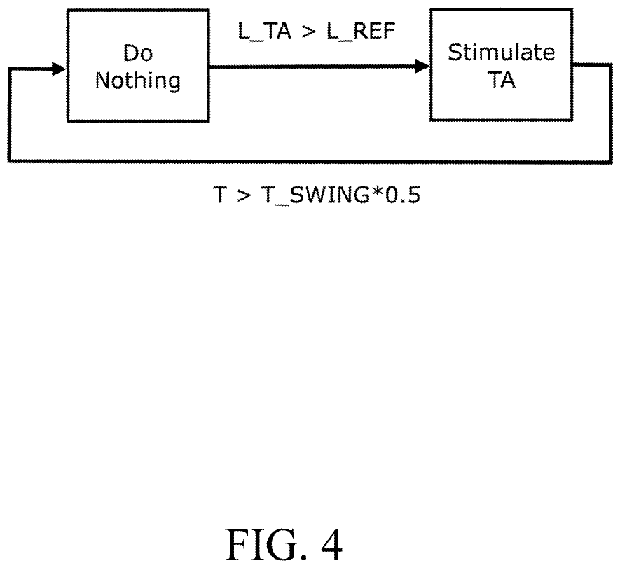

As described earlier, among the applications of the inventive concepts described herein is the use of combined stimulation and sensing. The hardware and method for stimulation and sensing can be different (for instance, functional electrical stimulation, optogenetics, magnetic fields, or direct nerve stimulation can be used for the stimulation, while any of the above strategies can be used for sensing). However, in some example embodiments, the sensing and stimulation are combined to minimize device cost, the number of insertion points, and foreign body reaction. As one example in which sensing and stimulation can be combined is the drop foot case as described earlier (see FIG. 2). In the case of drop foot, a pair of fine wires can be implanted percutaneously and fine-wire impedance myography can be used to sense the length and velocity of the muscle via onboard electronics. In addition, muscle EMG can be sensed to estimate muscle activation. By combining muscle length, speed and activation, muscle force can be estimated. Information about the length, velocity and/or force of the muscle can be used to determine the frequency, timing and magnitude of muscle stimulation via the same pair of fine wires, using a shared electronics board, which can either communicate wirelessly to a computer or house the computer and battery directly at the site at which the wires are inserted through the skin. In some embodiments, the length of a dorsiflexing muscle, for example the tibialis anterior (TA), can be used to determine the magnitude of stimulation of the that muscle, followed by a time delay that determines when to stop stimulating as a percentage of predicted swing phase time. This modality is shown in the following state transition diagram (FIG. 4).

FIG. 4 shows a State Transition Diagram for Drop foot Control via Combined Fine Wire Impedance Myography and Functional Electrical Stimulation in accordance with aspects of inventive concepts. In some embodiments, the tibialis anterior (TA) is monitored for length using fine wire impedance myography (fwIM), and when the measured length exceeds a reference length determined by calibration (for instance, observing the gait of a height and weight matched person or observing the movements of an unaffected leg), the TA is then stimulated during the early swing phase of gait using the same fine wires that are inserted into the TA for fwIM. The time is then monitored, and the stimulation is continued unless sufficient time has passed to suggest that the swing phase is almost or fully complete, where the percentage of swing time to stimulate is also determined similarly by calibration. Clinically, such a controller can enable a person with anterior muscle weakness to walk with sufficient dorsiflexion and foot clearance throughout the swing phase of walking.

In alternative embodiments, at least one of muscle length, speed or force, estimated using fwIM combined with EMG sensing, can serve as sensory inputs into a muscle stimulation controller in an updating, real time manner, for closed-loop muscle stimulation. For example, muscle length and speed can serve as inputs into a muscle stimulation controller to apply electrical stimulations that are updated in real time from these muscle state sensory inputs. If such a controller were applied to a drop foot patient, a biomimetic stretch reflex control can be used on the tibialis anterior during controlled plantar flexion, spanning from heel strike to forefoot strike during early stance, to eliminate forefoot slap against the walking surface. For such a TA controller applied during the controlled plantar flexion gait phase, muscle length and speed can be related to (and can serve as an input to a controller for) at least one of muscle stimulation frequency, pulse width or magnitude using linear or non-linear functional relationships. Here the greater the TA muscle stretch, and the velocity of stretch, the greater the muscle stimulation in real time. In other embodiments, the muscle stimulation controller can apply a positive force feedback reflex control on the soleus muscle for a person with posterior muscle compartment weakness. Here at least one of muscle stimulation frequency, pulse width or magnitude can be modulated in an updating manner under computer control during the controlled dorsiflexion and powered plantar flexion phases of gait using a positive force feedback reflex control. Using either a linear or nonlinear functional relationship, a positive force feedback function, with soleus force as the dominant sensory input, can determine the at least one of muscle stimulation frequency, pulse width or magnitude to achieve monotonically increasing muscle activations with increasing soleus muscle force.

Of course, any of these control instantiations can be used in other contexts, such as in the case of therapy, recovery, or movement restoration for a person with spinal cord injury or someone who has had a stroke. Further, additional sensing modalities, such as inertial measurement by IMUs, can be used to extend upon these control strategies.

In addition, in any of these cases, the central and/or peripheral nervous system can be used as a control signal, where the nervous system provides a control setpoint, the feedback provides information about the state of the muscle, and the stimulation controller modulates either a biological muscle or an exoskeletal synthetic actuator worn by the person in order to produce natural biomechanics. For example, if a person suffers from a weak soleus muscle, and this calf muscle weakness does not allow for a normal level of powered plantar flexion during the late stance phase of gait, a stimulation controller can be applied to augment the force and power of the calf muscle, and the biomechanical functionality of the person's gait. Here a linear or nonlinear functional relationship can be used to apply an exoskeleton actuator torque about the biological ankle during the powered plantar flexion phase of gait, wherein the exoskeleton torque monotonically increases with increasing calf muscle torque using a powered steering controller that augments calf muscle function.

### Generalizability

The use of the technology described herein can be extended to the sensing and stimulation of any muscle, including in both upper and lower extremities and in other locations within the body, such as smooth, skeletal, or cardiac muscle, including in movements of the digestive and pulmonary system and in movements of the eyes.

Any of the features described herein can be combined with any other of the features described herein to attain a more holistic and more accurate representation of the muscle state.

Of course, all of the example configurations described above can be applied to the measurement more generally of tissue or even of non-human, or of non-living, or of non-biological materials, especially of deformation of a material.

In various embodiments, devices, systems, and/or methods described herein may be replaceable. In various embodiments, devices, systems, and/or methods described herein may be configured to be recyclable.

In various embodiments, devices, systems, and methods described herein may be configured for user removal and/or application. In various embodiments, devices, systems, and/or methods described herein may be configured to alert a user when it is appropriately positioned to be applied to the user. In various embodiments, devices, systems, and/or methods described herein may use sensors (for example, ultrasound sensors) on the bottom to provide a user with a three-dimensional view of a target muscle on a display, and, in some embodiments, may highlight the projected insertion path of the fine wire strands into the muscle. In various embodiments, devices, systems, and/or methods described herein may display arrows to guide a user toward a correct positioning and/or application. In various embodiments, devices, systems, and/or methods described herein may alert a user when the tips of the fine wires are aligned so that they would be positioned on opposite ends of a fascicle in the target muscle. In various embodiments, devices, systems, and/or methods described herein may automatically insert wires into a user's muscle through the skin and lay down a small patch filled with electronics, a battery, and a processor. In various embodiments, devices, systems, and/or methods described herein may allow a user to view inserted wires using a mixed reality display. In various embodiments, devices, systems, and/or methods described herein may be applied to more than one muscle of a user's body.

In various embodiments, devices, systems, and/or methods described herein may be configured to be applied by a user in under ten minutes.

In various embodiments, devices, systems, and/or methods described herein may comprise strands of wires inserted through a user's skin into muscles for tracking biomechanics. In various embodiments, devices, systems, and/or methods described herein may allow a user to interact and receive interactive feedback with a mixed reality operating system during an augmented and/or mixed reality session by tracking muscle lengths and/or allowing stimulation of the muscles.

In various embodiments, devices, systems, and/or methods described herein may allow a user to control wearable and off-body robots. In various embodiments, devices, systems, and/or methods described herein may allow a user to control wearable and off-body robots via telepresence.

In various embodiments, devices, systems, and/or methods described herein may stimulate muscles to provide feedback for virtually-simulated forces during physical contact between more than one person. In various embodiments, devices, systems, and/or methods described herein may stimulate muscles to provide feedback for virtually-simulated forces during physical contact between shared physical or virtual items. In various embodiments, devices, systems, and/or methods described herein may be configured to be used during exercise.

The various features described herein may be used separately or in combination with each other.

While example embodiments have been particularly shown and described, it will be understood by those skilled in the art that various changes in form and details may be made therein without departing from the scope of the embodiments encompassed by the appended claims.

### Notable Relevant Prior Research in Bioimpedance Measurement

Tomiharu Yamaguchi, Emiyu Ogawa, and Akinori Ueno. “Short-Time Impedance Spectroscopy Using a Mode-Switching Nonsinusoidal Oscillator: Applicability to Biological Tissues and Continuous Measurement”. In: Sensors 21.21 (2021), p. 6951
Houman Mirzaalian Dastjerdi, Ramin Soltanzadeh, and Hossein Rabbani. “Designing and implementing bioimpedance spectroscopy device by measuring impedance in a mouse tissue”. In: Journal of medical signals and sensors 3.3 (2013), p. 187
Albert Ruiz-Vargas, Antoni Ivorra, and John William Arkwright. “Design, construction and validation of an electrical impedance probe with contact force and temperature sensors suitable for in-vivo measurements”. In: Scientific reports 8.1 (2018), pp. 1 -11
Justine Frija-Masson et al. “Accuracy of Smart Scales on Weight and Body Composition: Observational Study”. In: JMIR mHealth and uHealth 9.4 (2021), e22487
Roman Kusche and Martin Ryschka. “Combining bioimpedance and EMG measurements for reliable muscle contraction detection”. In: IEEE Sensors Journal 19.23 (2019), pp. 11687-11696
Seward B Rutkove. “Electrical impedance myography: background, current state, and future directions”. In: Muscle & Nerve: Official Journal of the American Association of Electrodiagnostic Medicine 40.6 (2009), pp. 936-946
Carl A Shiffman, H Kashuri, and R Aaron. “Electrical impedance myography at frequencies up to 2 MHz”. In: Physiological measurement 29.6 (2008), S345
Olumuyiwa T Ogunnika et al. “A handheld electrical impedance myography probe for the assessment of neuromuscular disease”. In: 2008 30th Annual International Conference of the IEEE Engineering in Medicine and Biology Society. IEEE. 2008, pp. 3566-3569
Carl A Shiffman, Ronald Aaron, and Seward B Rutkove. “Electrical impedance of muscle during isometric contraction”. In: Physiological Measurement 24.1 (2003), p. 213

What is claimed is:

1. A muscle interface, comprising:
an implant comprising a plurality of fine wires configured to be disposed in muscle tissue and to detect an electrical impedance at the muscle tissue; and
a processor configured to determine a state of the muscle tissue based on the detected electrical impedance.

2. The muscle interface of claim 1, wherein the implant is configured to detect at least two electrical impedances at the muscle tissue.

3. The muscle interface of claim 2, wherein the implant is configured to detect an electrical impedance between each combination of two fine wires of the plurality of fine wires.

4. The muscle interface of claim 1, wherein the determined state comprises at least one of length, velocity, or deformation of the muscle tissue.

5. The muscle interface of claim 1, wherein the implant is further configured to detect an electromyographic signal at at least one wire of the plurality, at an additional wire of the implant, or at a combination thereof.

6. The muscle interface of claim 5, wherein the processor is further configured to determine an activation level of the muscle tissue based on the detected electromyographic signal.

7. The muscle interface of claim 1, further comprising a controller electrically coupled to the processor.

8. The muscle interface of claim 1, wherein the implant is further configured to electrically stimulate the muscle tissue via at least one wire of the plurality, at an additional wire of the implant, or at a combination thereof.

9. The muscle interface of claim 7, wherein the controller is further configured to generate a muscle stimulation signal for the implant based on the determined state.

10. The muscle interface of claim 7, wherein the controller is further configured to generate a control signal for an actuator of a wearable robotic device based on the determined state.

11. A method of interfacing with a muscle, comprising:
detecting an electrical impedance at a muscle tissue with an implant comprising a plurality of fine wires disposed in the muscle tissue; and
determining a state of the muscle tissue based on the detected electrical impedance.

12. The method of claim 11, further comprising detecting at least two electrical impedances at the muscle tissue.

13. The method of claim 12, further comprising detecting an electrical impedance between each combination of two fine wires of the plurality of fine wires.

14. The method of claim 11, wherein the determined state comprises at least one of length, velocity, or deformation of the muscle tissue.

15. The method of claim 11, further comprising detecting an electromyographic signal at at least one wire of the plurality, at an additional wire of the implant, or at a combination thereof.

16. The method of claim 15, further comprising determining an activation level of the muscle tissue based on the detected electromyographic signal.

17. The method of claim 11, further comprising electrically stimulating the muscle tissue via at least one wire of the plurality, at an additional wire of the implant, or at a combination thereof.

18. The method of claim 11, further comprising generating a muscle stimulation signal for the implant based on the state.

19. The method of claim 11, further comprising generating a control signal for an actuator of a wearable robotic device based on the state.

20. A method for controlling drop foot, comprising:
monitoring a length of a tibialis anterior using a fine wire impedance myography device, the device comprising fine wires;
determining if the length of the tibialis anterior exceeds a reference length;
stimulating the tibialis anterior during an early swing phase of gait using the fine wires of the fine wire impedance myography device; and
ending the stimulation when the swing phase is more than a given percentage complete.
